(19) United States
(12) Patent Application Publication
(10) Pub. No.: US 2006/0167465 A1
(43) Pub. Date: Jul. 27, 2006

(54) System for facilitating attachment of a delivery instrument with a bone screw

(76) Inventor: Erasmo Lopez, Abington, MA (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 11/339,871

(22) Filed: Jan. 26, 2006

Related U.S. Application Data
(60) Provisional application No. 60/647,473, filed on Jan. 27, 2005.

(51) Int. Cl. A61F 2/36

(52) U.S. Cl. 606/104

(57) ABSTRACT

A system for facilitating attachment of a bone anchor with an instrument includes an alignment device having an anchor opening sized and shaped to receive a bone anchor in a predefined orientation and a guide tube connectable to the alignment device. The guide tube may have a lumen for receiving an instrument for the bone anchor. The lumen of the guide tube may be aligned with the anchor opening in the alignment device upon connection of the guide tube to the alignment device. An instrument may be advanced into the lumen of the guide tube in a first orientation. The instrument can be connected to the bone anchor by moving the instrument to a second orientation.

### REFERENCE TO RELATED APPLICATIONS

The present application claims priority to U.S. Provisional Patent Application No. 60/647,473, filed Jan. 27, 2005, incorporated herein by reference.

### BACKGROUND

Recently, the trend in spinal surgery has been moving toward providing minimally invasive devices and methods for implanting spinal implants, such as bone anchors and spinal fixation devices in connection with, for example, spinal fusion procedures. Minimally invasive devices and methods provide many advantages over conventional procedures, including limiting the trauma to surrounding tissue. In such minimally invasive procedures, the surgeon's ability to visualize and control the position of a spinal implant relative to the spinal anatomy is limited because of the relatively small size of the access incision. As a result, specialized minimally invasive surgical instruments have been design to allow, for example, a bone anchor and other implants to be delivered through a minimally invasive incision, such as a percutaneous stab incision. In the case of bone anchors, such as pedicle screws, it is important that the minimally invasive delivery instrument be properly aligned and oriented with the bone anchor to facilitate proper placement of the bone anchor through the minimally invasive incision.

### SUMMARY

Disclosed herein are systems and methods for facilitating the attachment of an instrument to a bone anchor. The systems and methods disclosed herein are particularly suited to facilitating the connection of a minimally invasive delivery instrument to a bone anchor and insuring that the bone anchor is properly aligned and oriented relative to the delivery instrument.

In accordance with one exemplary embodiment, a system for facilitating attachment of a bone anchor with an instrument may comprise an alignment device including an anchor opening sized and shaped to receive a bone anchor in a predefined orientation and a guide tube having a lumen for receiving a delivery instrument for the bone anchor. The lumen of the guide tube may be aligned with the anchor opening in the alignment device.

An exemplary method of connecting an instrument to a bone anchor may comprise inserting a bone anchor into an anchor opening of an alignment device, connecting a guide tube to the anchor device, positioning a delivery instrument into the lumen of the guide tube in a first orientation, and connecting the delivery instrument to the bone anchor by moving the delivery instrument to a second orientation.

### BRIEF DESCRIPTION OF THE FIGURES

These and other features and advantages of the systems and methods disclosed herein will be more fully understood by reference to the following detailed description in conjunction with the attached drawings in which like reference numerals refer to like elements through the different views. The drawings illustrate principles of the systems and methods disclosed herein and, although not to scale, show relative dimensions.

FIG. 1 is a perspective view of an exemplary embodiment of an alignment device;

FIG. 2 is a front view of the alignment device of FIG. 1;

FIG. 3 is a top view of the alignment device of FIG. 1;

FIG. 4 is a side view of the alignment device of FIG. 1;

FIG. 5 is a cross sectional view of the alignment device of FIG. 1, taken along the line A-A of FIG. 4;

FIG. 6 is a perspective view of an exemplary embodiment of a guide tube;

FIG. 7 is a bottom view of the guide tube of FIG. 6;

FIG. 8 is a top view of the guide tube of FIG. 6;

FIG. 9 is a side view of the guide tube of FIG. 6;

FIG. 10 is a cross sectional view of the guide tube of FIG. 6, taken along the line B-B of FIG. 9;

FIG. 11 is a cross sectional view of the guide tube of FIG. 6, taken along the line C-C of FIG. 10;

FIG. 12A is a perspective view of an exemplary bone anchor assembly;

FIG. 12B is a side view of the bone anchor assembly of FIG. 12A;

FIG. 13 is a perspective view of the alignment device of FIG. 1, illustrating the bone anchor assembly of FIGS. 12A-B positioned within an anchor hole of the alignment device;

FIG. 14 is a top view of the alignment device of FIG. 1, illustrating the bone anchor assembly of FIGS. 12A-B positioned within an anchor hole of the alignment device;

FIG. 15 is a side view of the alignment device of FIG. 1, illustrating the bone anchor assembly of FIGS. 12A-B positioned within an anchor hole of the alignment device;

FIG. 16 is a cross sectional view of the alignment device of FIG. 1 taken along the line D-D of FIG. 15, illustrating the bone anchor assembly of FIGS. 12A-B positioned within an anchor hole of the alignment device;

FIG. 17 is a side view of the alignment device of FIG. 1, illustrating the bone anchor assembly of FIGS. 12A-B positioned within an anchor hole of the alignment device and the guide tube of FIG. 6 connected to the alignment device;

FIG. 18 is a cross sectional view of the alignment device of FIG. 1 taken along the line E-E of FIG. 17, illustrating the bone anchor assembly of FIGS. 12A-B positioned within an anchor hole of the alignment device and the guide tube of FIG. 6 connected to the alignment device;

FIG. 19 is a cross sectional view of the alignment device of FIG. 1 taken along the line E-E of FIG. 17, illustrating the bone anchor assembly of FIGS. 12A-B positioned within an anchor hole of the alignment device, the guide tube of FIG. 6 connected to the alignment device, and a delivery instrument positioned in the guide tube in a first orientation;

FIG. 20 is a detailed cross sectional view of the system of FIG. 19, illustrating the alignment device, the guide tube, and the delivery instrument in cross section.

FIG. 21A is a cross sectional view of the alignment device of FIG. 1 taken along the line E-E of FIG. 17, illustrating the bone anchor assembly of FIGS. 12A-B positioned within an anchor hole of the alignment device, the guide tube of FIG. 6 connected to the alignment device, and a delivery instrument positioned in the guide tube in a first orientation;

FIG. 21B is a detailed cross sectional view of the system of FIG. 21A, illustrating the alignment device and the guide tube in cross section;

FIG. 22 is a cross sectional view of the alignment device of FIG. 1 taken along the line E-E of FIG. 17, illustrating the bone anchor assembly of FIGS. 12A-B positioned within an anchor hole of the alignment device, the guide tube of FIG. 6 connected to the alignment device, and a delivery instrument positioned in the guide tube and rotated to a second orientation;

FIG. 23 is a detailed cross sectional view of the system of FIG. 22, illustrating the alignment device, the guide tube, and the delivery instrument in cross section.

FIG. 24 is a detailed cross sectional view of the system of FIG. 22, illustrating the alignment device and the guide tube in cross section;

FIG. 25 is a perspective view of a delivery instrument connected to the bone anchor assembly of FIGS. 12A-B;

FIG. 26 is a perspective view of the delivery instrument and bone anchor assembly of FIG. 25, illustrating an anchor driver positioned within the delivery instrument and engaging the bone anchor assembly,

FIG. 27 is a perspective view of another exemplary embodiment of an alignment device and guide tubes, illustrating a guide tube pivoted to a first position and a guide tube pivoted to a second position;

FIG. 28 is a partially exploded perspective view of the alignment device and guide tubes of FIG. 27;

FIG. 29 is a perspective vice of the alignment device of FIG. 27; and

FIG. 30 is a perspective view of a guide tube of FIG. 27.

### DETAIL DESCRIPTION

Certain exemplary embodiments will now be described to provide an overall understanding of the principles of the structure, function, manufacture, and use of the systems and methods disclosed herein. One or more examples of these embodiments are illustrated in the accompanying drawings. Those of ordinary skill in the art will understand that the systems specifically described herein and illustrated in the accompanying drawings are non-limiting exemplary embodiments and that the scope of the present invention is defined solely by the claims. The features illustrated or described in connection with one exemplary embodiment may be combined with the features of other embodiments. Such modifications and variations are intended to be included within the scope of the present invention.

The articles “a” and “an” are used herein to refer to one or to more than one (i.e. to at least one) of the grammatical object of the article. By way of example, “an element” means one element or more than one element.

The terms “comprise,” “include,” and “have,” and the derivatives thereof, are used herein interchangeably as comprehensive, open-ended terms. For example, use of “comprising,” “including,” or “having” means that whatever element is comprised, had, or included, is not the only element encompassed by the subject of the clause that contains the verb.

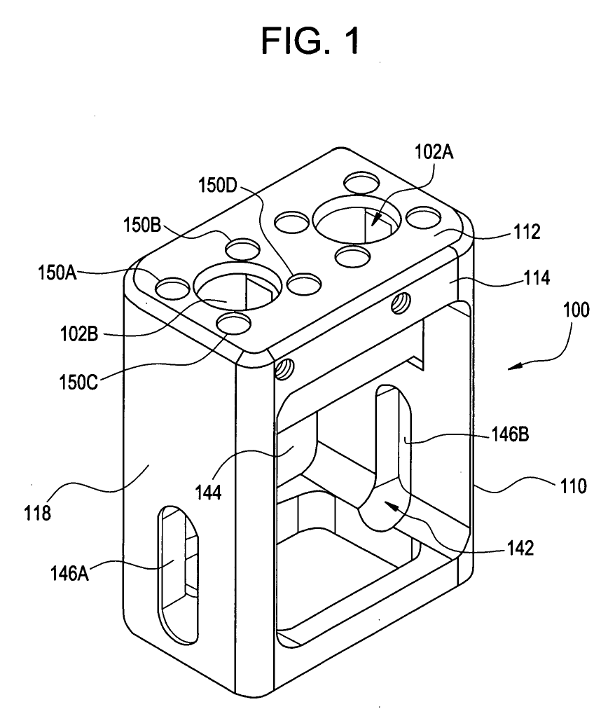

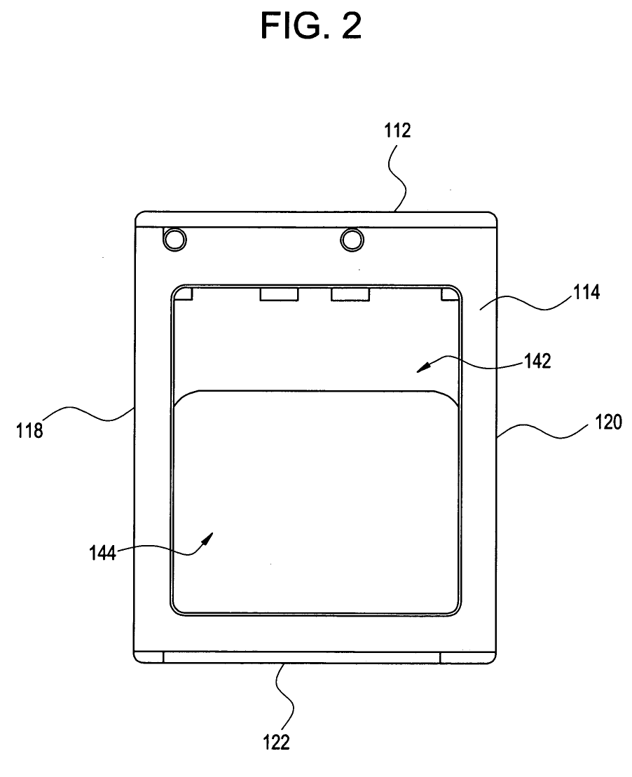

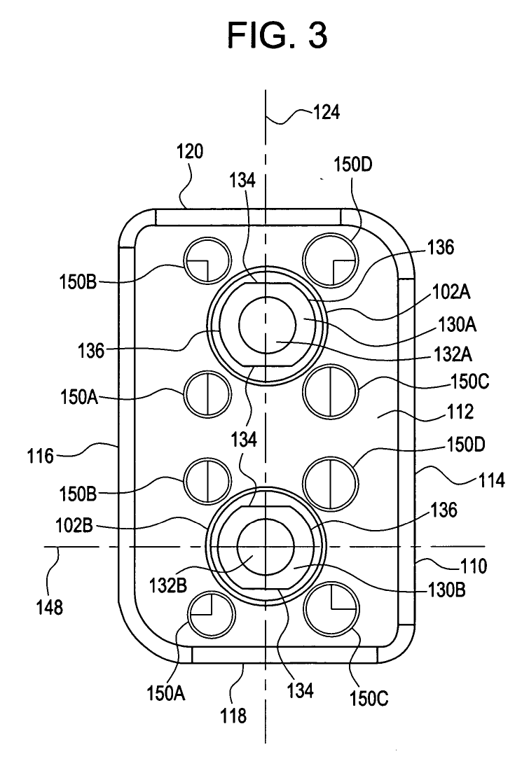

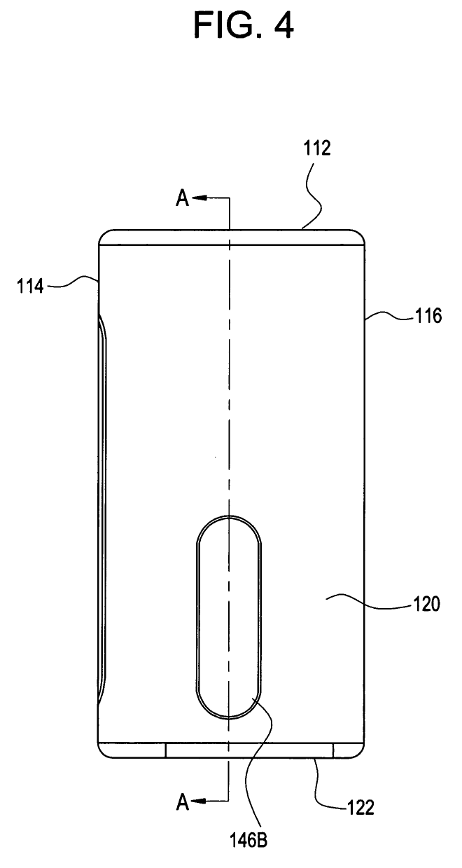

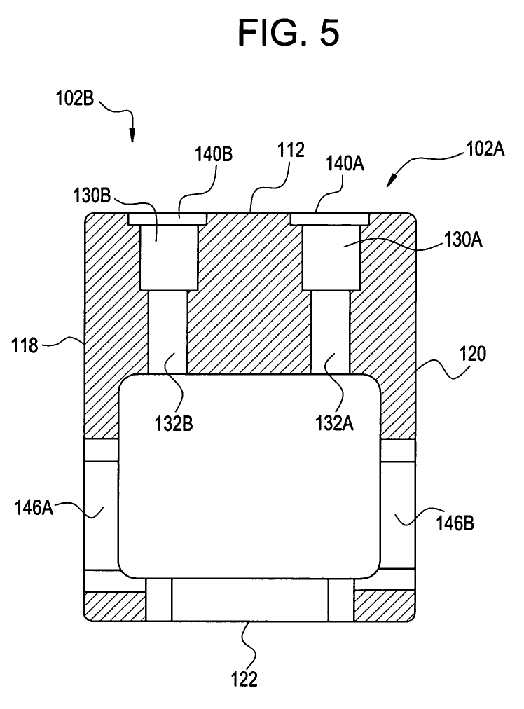

FIGS. 1-5 illustrate an exemplary embodiment of an alignment device 100 for facilitating alignment and connection of a bone anchor with an instrument. The alignment device 100 is particularly suited to facilitate the connection of a polyaxial bone screw with an instrument such as a minimally invasive delivery instrument for the bone anchor. However, the exemplary alignment device may be employed to facilitate the connection of any type of bone anchor, including, for example, monoaxial bone screws and hooks, with any type of instrument, including, for example, a spinal approximator, a bone anchor driver, a compression/distraction instrument, or any other instrument used in spinal surgical procedures.

The exemplary alignment device 100 includes one or more anchor openings 102 for receiving a respective bone anchor. In the illustrated embodiment, the alignment device 100 includes a first anchor opening 102A and a second anchor opening 102B, although any number of anchor openings, including a single anchor opening, may be provided. The aliment device 100 comprises a block-shaped body 110 having the following planar surfaces: a top (proximal) surface 112, a front surface 114, a rear surface 116, opposing side surfaces 118, 120, and a bottom (distal) surface 122. The alignment device 100 is not limited to the illustrated block-shape; the alignment device can be any suitable shape, including for example, rectilinear, cylindrical, elliptical. The alignment device 100 may be constructed of any material suitable for medical instruments, including, for example, metal, such as stainless steel, polymers, ceramics, and composites thereof.

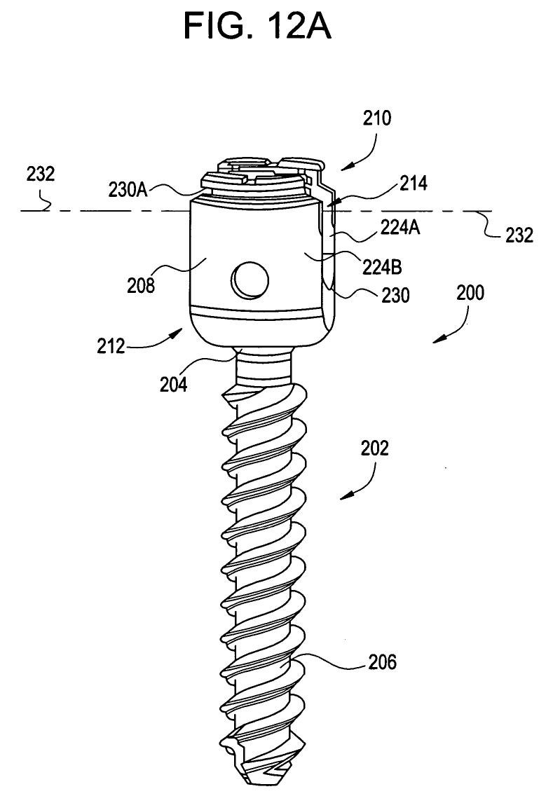

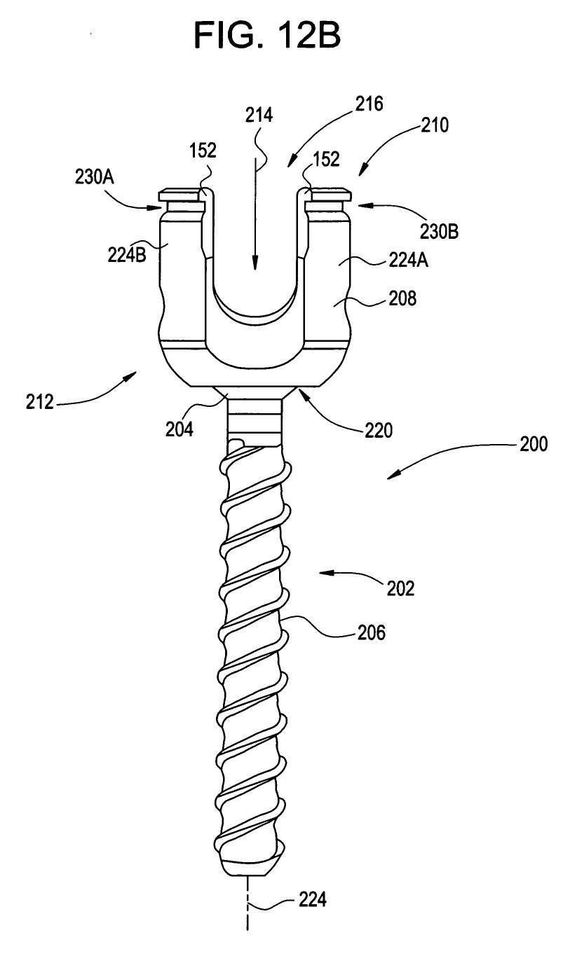

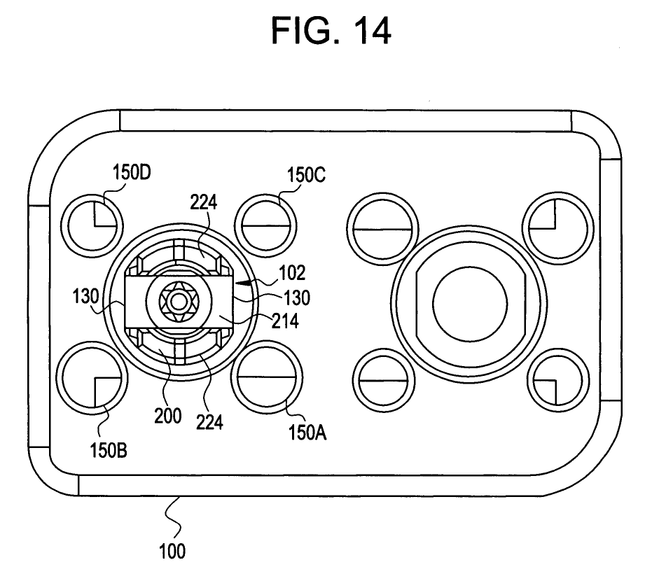

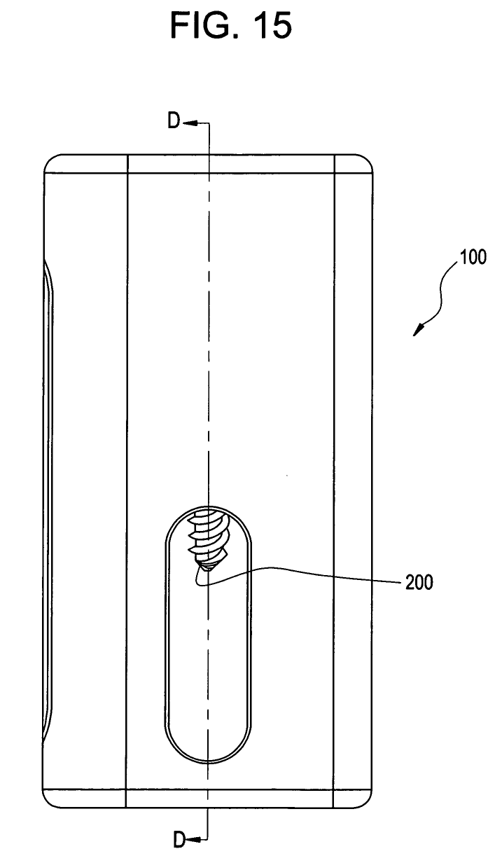

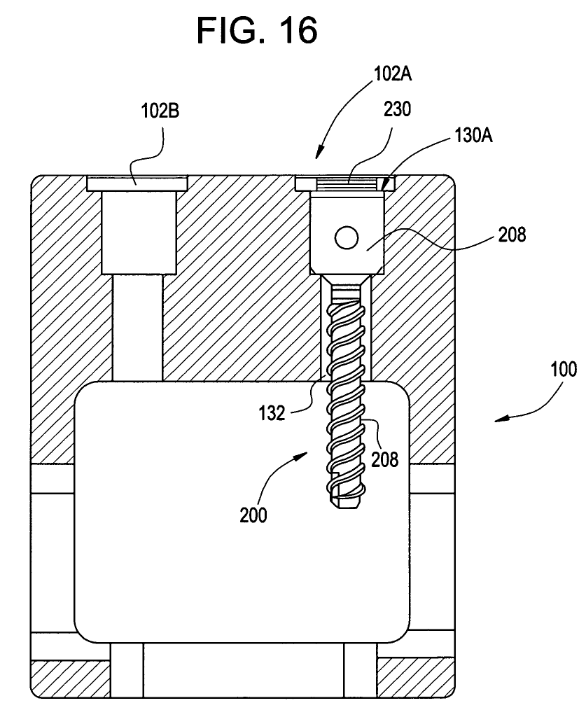

The anchor openings 102 may be formed in the top surface 112 of the alignment device 100. One or more of the anchor openings 102 of the alignment device 100 may be sized and shaped to receive the bone anchor in a predefined orientation. For example, in the case of a bone anchor having a receiving member defining a slot for receiving a spinal fixation element, such as a spinal rod, a plate, or a tether, an anchor opening may sized and shaped to orient the slot of the bone anchor in a predefined orientation. As discussed in more detail below in connection with FIGS. 12A-16, the anchor openings 102A and 102B of the illustrated alignment device 100 are sized and shaped to orient the slot of a bone anchor parallel to a first axis 124 of the alignment device 100. In particular, each anchor opening 102A,B includes a proximal portion 130 for receiving the receiving member of a bone anchor and a distal portion 132 for receiving a bone engaging portion of the bone anchor. In the exemplary embodiment, the proximal portion 130 of the anchor opening 102 is sized and shaped to orient the slot of the receiving member of the bone anchor parallel to a first axis 124 of the alignment device 100. For example, the proximal portion 130 may have a size and shape corresponding to the size and shape of the receiving member of the bone anchor. In the case of the exemplary polyaxial bone screw assembly 200 described below in connection with FIGS. 12A-B, the proximal portion 130 of each anchor opening 102A,B may include a pair of opposed planar side walls 134 spaced apart by a pair of opposed arcuate side walls 136. The opposed planar side walls 134 are oriented parallel to each other and parallel to a second axis 148 of the alignment device 100, which is itself oriented perpendicular to the first axis 124 of the alignment device 100.

The distal portion 132 of the anchor opening 102 may be sized and shaped to receive a bone engaging portion of the bone anchor. In the case of the exemplary polyaxial bone screw assembly 200 described below in connection with FIGS. 12A-B, distal portion 132 is generally cylindrical in shape having a diameter greater than a major diameter of the threads provided on the externally threaded shank of the bone screw assembly 200. In alternative embodiments, the anchor opening 102 of the alignment device 100 may include only one portion, for example the proximal portion. The anchor opening 102 may include a counter sink 140 to facilitate placement of the distal end of an instrument in contact with a proximal end of a bone anchor positioned in the anchor opening 102.

The alignment device 100 may include one or more windows to allow a bone anchor to be visualized when the bone anchor is positioned in an anchor opening 102 of the alignment device 100 and to reduce the weight of the alignment device 100. The number and position of the window(s) may be varied depending on, for example, the type of bone anchor, and the material, the size, and the shape of the alignment device. In the illustrated exemplary embodiment, for example, the front surface 114 has a front window 142 that communicates with a rear window 144 formed in the rear surface 116 of the alignment device 100. In addition, each side surface 118, 120 may include a side window 146A, 146B.

The top surface 112 of the alignment device 100 may include one or more connection openings 150 arranged about an anchor opening 102 to facilitate connection of a guide tube, described below, or an instrument to the alignment device 100. In the illustrated exemplary embodiment, the connection openings 150 each may receive an alignment post 350 extending from the distal end of a guide tube 300. The connection openings 150 may be sized, shaped, and/or arranged to facilitate positioning of the guide tube, or an instrument, to the alignment device 100 in a predefined orientation. For example, in the illustrated exemplary embodiment, the plurality of connection openings include a first connection opening 150A and a second connection opening 150B that are generally circular in shape having a first diameter and a third connection opening 150C and a fourth connection opening 150D having a second diameter distinct from the first diameter. For example, the first diameter may be less than the second diameter, as in the illustrated embodiment, or the first diameter may be greater than the second diameter. Any number of connection openings 150 may be provided, including a single connection opening. In alternative exemplary embodiments, the shape of the connection openings 150 arranged about an anchor opening may be varied to facilitate positioning of the guide tube in a predefined orientation. Any shape may be employed, included circular, rectilinear, elliptical, or triangular.

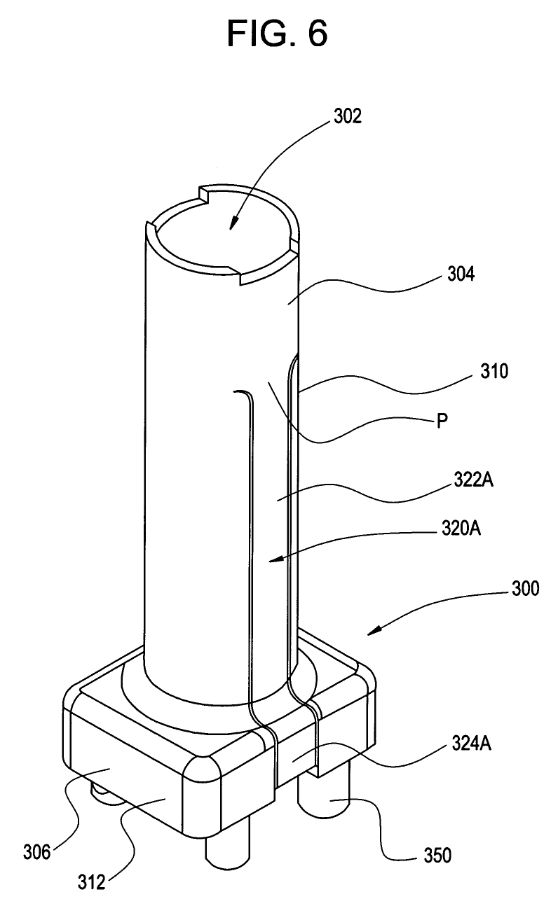

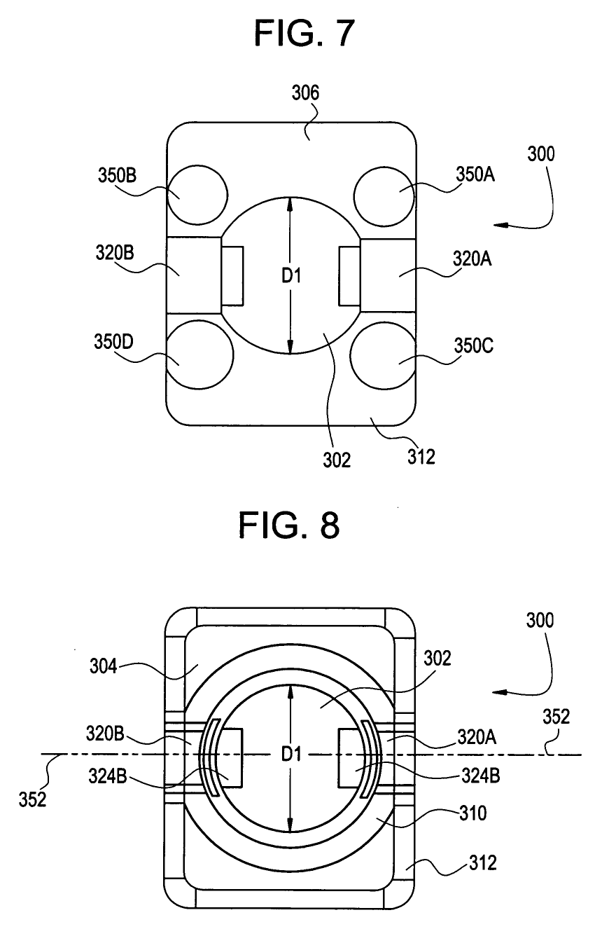

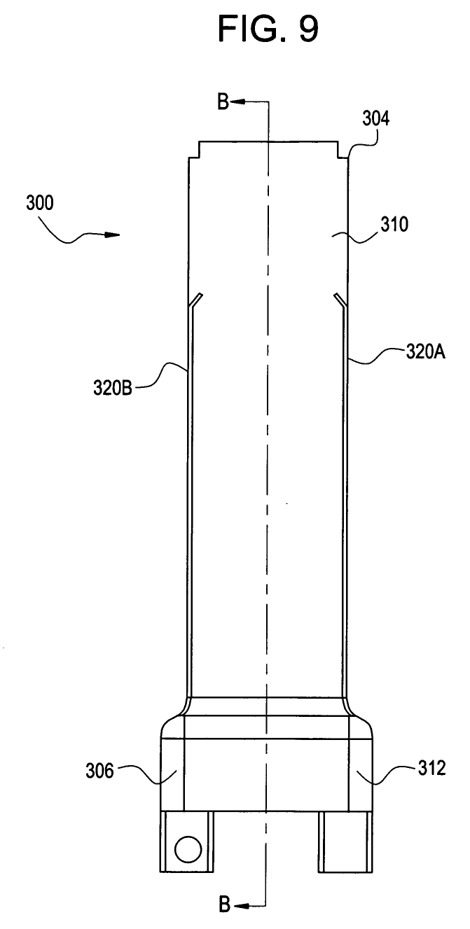

FIGS. 6-11 illustrate an exemplary embodiment of a guide tube 300 connectable to an alignment device, such as the alignment device 100, described above, to facilitate alignment of an instrument with a bone anchor positioned in an anchor opening of the alignment device. The guide tube 300 may have a lumen 302 extending from a proximal end 304 of the guide tube 300 to a distal end 306 of the guide tube 300. In the illustrated embodiment, the guide tube 300 includes a generally cylindrical section 310 and a base section 312 positioned distal to the cylindrical section 310 proximate the distal end 306 of the guide tube 300. The cylindrical section 310 may be circular in cross-section, as illustrated, or may have other cross-sectional shapes, including, rectilinear, elliptical, polygonal, or triangular. The base section 306 may be generally block-shaped as illustrated or may have other shapes suitable to stabilize the guide tube 300 when the guide tube 300 is positioned on the alignment device. The guide tube 300 may be constructed of any material suitable for medical instruments, including, for example, metal, such as stainless steel, polymers, ceramics, and composites thereof.

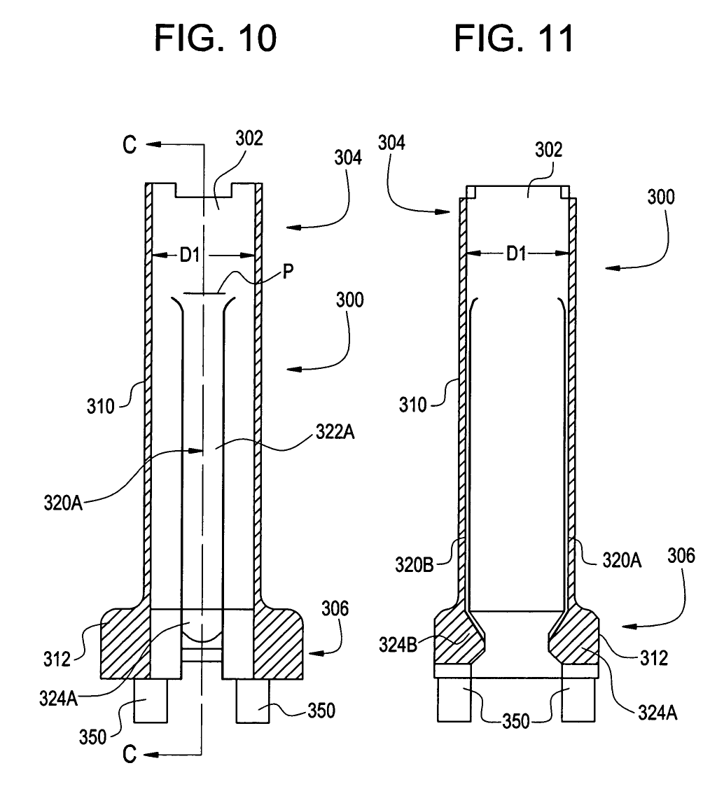

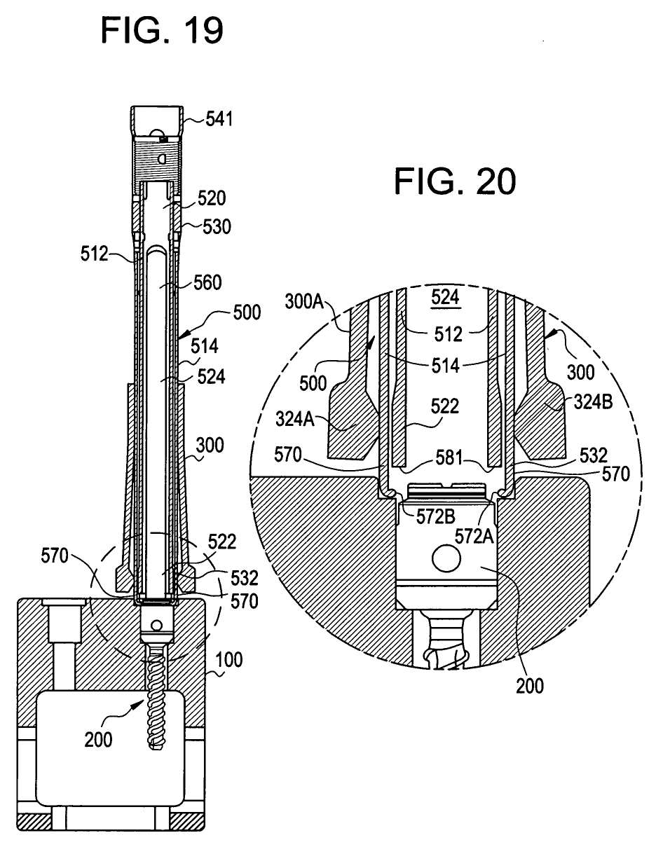

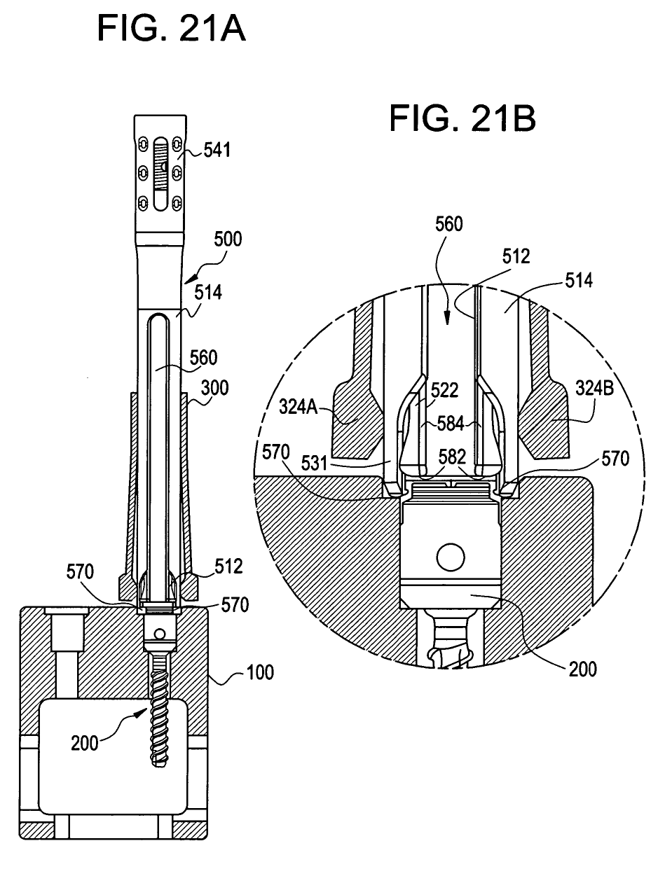

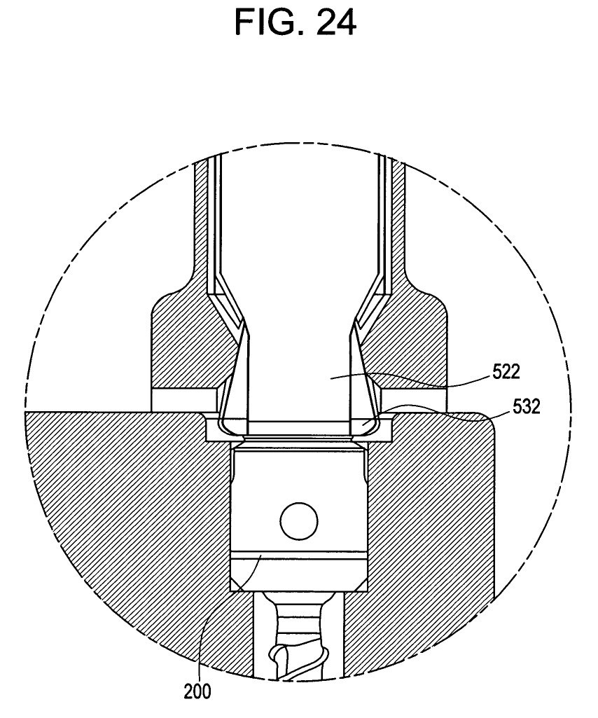

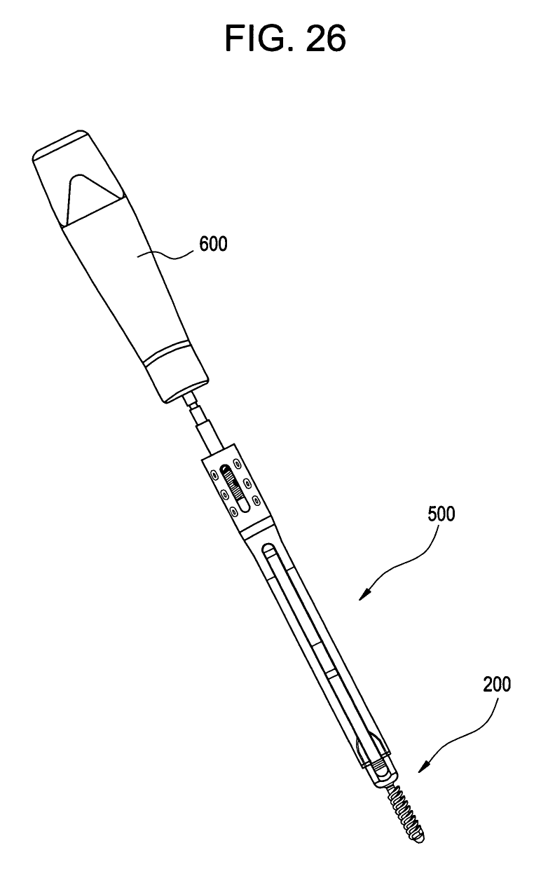

In the illustrated exemplary embodiment, the proximal section of the lumen 302 of the guide tube 300 is defined by the walls of the cylindrical section 310 of the guide tube 300 and the distal section of the lumen 302 is defined by a cylindrical passage formed in the base section 312. As illustrated in FIGS. 10 and 11, the lumen 302 is open at the proximal end 304 of the guide tube 300 and is open at the distal end 306 of the guide tube 300. Continuing to refer to FIGS. 6-11, the diameter D1 of the lumen 302, as defined by the cylindrical section 310 and the base section 312, is sized to receive an instrument therethrough. For example, the diameter D1 of the lumen 302 may be greater than or equal to the outer diameter of an instrument to be guided and connected to a bone anchor positioned in the alignment device. In the case of the percutaneous access device 500 described below in connection with FIGS. 19-26, the diameter D1 of the lumen 302 is greater than the outer diameter of the percutaneous access device 500 to allow the percutaneous access device 500 to be delivered through the lumen 302 into engagement with a bone anchor positioned in the alignment device 100.

The guide tube 300 may include one or more tabs 320 for engaging an instrument positioned in the lumen 302 of the guide tube 300 and controlling the orientation of the instrument in the guide tube 300. In the illustrated exemplary embodiment, the guide tube 300 has a pair of diametrically opposed tabs 320A, 320B. The tabs 320A,B include an elongated section 322, which extends distally in a direction parallel to the longitudinal axis of the guide tube 300, and a engagement section 324 at the terminus of the elongated section 322. The engagement section 324 projects radially inward into the lumen 302 of the guide tube 300 to engage an instrument positioned in the lumen 302. In the illustrated exemplary embodiment, the tabs 320A, B are formed from the walls of the guide tube 300. The proximal end of the elongated section 322 of each tab 320A,B is connected to the cylindrical section 310 of the guide tube 300 to define a pivot axis P about which each tab may pivot from a first position to a second position. In the first position, illustrated in FIG. 11, the elongated section 322 of each of the tabs 320A,B is generally aligned with the walls of the guide tube 300 and the engagement section 324 of each of the resilient tabs projects radially inward into the lumen 302 of the guide tube. In the second position, each tab 320 is displaced radially outward, as illustrated in FIGS. 19-21, such that the engagement section 324 of each tab 320 may contact an outer surface of an instrument positioned within the lumen 302. In the second position, the engagement section 324 of each tab 320 may be displaced at least partially and, in some embodiments, completely external to the lumen 302 of the guide tube 300. The tabs 320A,B may be constructed from a flexible, resilient material to facilitate displacement of the tabs between the first and second positions. The tabs 320A,B may be biased to the first position.

The base section 312 of the guide tube 300 may include one or more alignment posts 350 that extend distally from the base section 312 to facilitate engagement of the guide tube to an alignment device, such as the alignment device 100 described above. Any number of alignment posts 350, including one alignment post, may be provided in any position suitable to facilitate engagement of the guide tube to an alignment device. In the illustrated exemplary embodiment, the base section 312 includes four alignment posts 350A-D positioned about the periphery of the base section 312. The alignment posts 350 may have a size, shape, and/or position to facilitate alignment of the guide tube 300 in a predefined orientation relative to the alignment device. For example, in the illustrated exemplary embodiment, the alignment posts 350A-D are sized, shaped, and positioned to fit within the connection openings 150A-D provided about an anchor opening 102 of the alignment device 100 to facilitate alignment of the lumen 302 of the guide tube 300 with an anchor opening 102. Moreover, the alignment posts 350, in cooperation with the connection openings 150, may be configured to position the guide tube 300 in a predefined orientation relative to an alignment device. In the illustrated exemplary embodiment, for example, the alignment posts 350A-D are configured to orient the guide tube 300 relative to the alignment device 100 such that a tab axis 352 intersecting the tabs 320A,320B is oriented parallel to the first axis 124 of the alignment device 100. In particular, alignment posts 350A-D may be sized and shaped analogously to the size and shape of the connection openings 150A-D, respectively. The first alignment post 350A and the second alignment post 350B may be generally cylindrical in shape having first diameter and the third alignment post 350C and fourth alignment post 350D may be generally cylindrical in shape having a second diameter that is greater than the first diameter. In alternative exemplary embodiments, the shape of the alignment posts 350 may be varied to facilitate the positioning of the guide tube 300 in a predefined orientation relative to the alignment device 100. Any cross-sectional shape may be employed, included circular, rectilinear, elliptical, or triangular.

The operation of the alignment device 100 and with the guide tube 300 will be described in connection with a polyaxial bone screw assembly, such as a pedicle screw, and a delivery instrument, such as percutaneous access device for delivering a pedicle screw to the pedicle of a vertebra. One skilled in the art will appreciate that the exemplary alignment device 100 and guide tube 300 can be used with any type of bone anchor and any type of instrument. FIGS. 12A-B illustrate an exemplary embodiment of a polyaxial bone screw assembly 200 designed to be implanted into the pedicle of a vertebra. Exemplary bone anchor assembly 200 includes a bone screw 202, such as a pedicle screw, having a proximal head 204 and a distal bone engaging portion 206, which in the illustrated exemplary embodiment is an externally threaded screw shank. The exemplary bone screw assembly 200 also includes a receiving member 208 that is configured to receive and couple a spinal fixation element, such as a spinal rod or spinal plate, to the bone anchor assembly 200.

The receiving member 208 may be coupled to the bone anchor 202 in any well-known conventional manner. For example, the bone anchor assembly may be poly-axial, as in the present exemplary embodiment in which the bone anchor 202 may be adjustable to multiple angles relative to the receiving member 208, or the bone anchor assembly may be mono-axial, e.g., the bone anchor 202 is fixed relative to the receiving member 208. An exemplary poly-axial bone screw is described U.S. Pat. No. 5,672,176, incorporated herein by reference. In mono-axial embodiments, the bone anchor 202 and the receiving member may be coaxial or may be oriented at angle with respect to one another. In poly-axial embodiments, the bone anchor may biased to a particular angle or range of angles to provide a favored angle the bone anchor. Exemplary favored-angle bone screws are described in U.S. Patent Application Publication No. 2003/0055426 and U.S. Patent Application Publication No. 2002/0058942, both of which are incorporated herein by reference.

The receiving member 208 of the illustrated exemplary embodiment includes a proximal end 210, a distal end 212, and a recess or slot 214 for receiving a spinal fixation element such as a spinal rod. The proximal end 210 of the receiving member 208 has a first bore 216. The recess 214 communicates with the first bore 216 such that a spinal fixation element may be positioned through the first bore 216 into the recess 214. The distal end 212 has a second bore 220 opposite the second bore 216. The second bore 220 is designed to receive the head 204 of the bone anchor 202 to couple the bone anchor 202 to the receiving member 208. In the illustrated exemplary embodiment, the head 204 is seated within the second bore 220. As the exemplary illustrated embodiment of the bone anchor assembly is poly-axial, the bone anchor 202 is free to rotate relative to the receiving member 208 such that the longitudinal axis 224 of the bone anchor 202 is positionable at an angle relative to the bore axis of the second bore 220. The second bore 220 may be conically shaped to facilitate adjustment of the bone anchor 202 relative to the receiving member 208. In the exemplary embodiment, the receiving member 208 has a generally U-shaped cross-section defined by two legs 224A and 224B separated by recess 114. Each leg 224A, 224B is free at the proximal end 210 of the receiving member 208. The sides 230 of each leg 224A, 224B are generally planar extending in a direction generally transverse to a recess axis 232 defined by the recess 214.

The receiving member 208 may be configured to receive a closure mechanism that locks a spinal fixation element within the recess 214. The closure mechanism may be a cap that is advanceable through the first bore 216 of the receiving member 208 and seats against the spinal fixation element. For example, the cap may have external threads that engage internal threads provided in the receiving member 208, e.g., on the legs 224A,B, as in the exemplary embodiment. Any type of conventional closure mechanism may be employed, including, for example, non-threaded caps, multi-component closure mechanisms, and/or external caps.

The receiving member 208 of the exemplary bone anchor assembly 200 is configured to be releasably connected to an instrument such as the exemplary percutaneous access device 500 described below. For example, the receiving member 208 may include at least one groove 230 that is configured to receive a portion of an instrument to releasably connect the instrument to the bone anchor assembly. The size, shape, position, and number of grooves can be varied depending on, for example, the instrument employed and the type of connection desired. In certain embodiments, for example, at least one arcuate groove 230 may be provided on an exterior surface of the proximal end 210 of the receiving member 208. In the illustrated exemplary embodiment, each leg 224A and 224B may be provided with an arcuate groove 230A, 230B, respectively, at the free, proximal end of the leg 224A, 224B. The grooves 230A, 230B may extend about a portion or all of the circumference of the proximal end of each leg 224A, 224B. Each groove 230A, 230B may have size and shape that is complementary in size and shape to a projection provided on the instrument. For example, in the illustrated exemplary embodiment, the each groove 230A, 230B may be arcuate and may have a cross-section complementary to the cross-section of a projection 572A, 572B provided on the tabs 570A,570B of the outer sleeve 514 of the percutaneous access device 500, as illustrated in FIG. 20.

An exemplary percutaneous access device 500 for delivering a bone anchor, such as bone anchor assembly 200, to a vertebra is illustrated in FIGS. 19-26. The exemplary percutaneous access device 500 can facilitate the delivery and implanting of a bone anchor, such as the exemplary bone anchor assembly 200 illustrated and described above, into bone, in particular, one or more vertebral bodies of the spine. In particular, the exemplary percutaneous access device 500 can facilitate the delivery and implanting of a bone anchor in a minimally invasive manner and can provide a percutaneous pathway between a skin incision in the patent and the bone anchor that may be used to deliver components of the bone anchor, such as the closure mechanism, one or more fixation elements, and/or instruments to the bone anchor. The percutaneous access device 500 is preferably adapted to be introduced through a minimally invasive percutaneous incision, which is a relatively small incision that typically has a length less than the diameter or width of the device being inserted therethrough. The exemplary percutaneous access device 500 and the exemplary bone anchor assembly 200 are described in U.S. Patent Application Publication No. 2005/0131408, which is incorporated herein by reference.

The exemplary percutaneous access device 500 includes an inner tube 512 and an outer tube 514 disposed about at least a portion of the inner tube 512. In the illustrated exemplary embodiment, the outer tube 514 is coaxially disposed about the inner tube 512 such that the inner tube 512 and the outer tube 514 share a common longitudinal axis. One skilled in the art will appreciate, however, that the outer tube 514 and inner tube 512 need not be coaxially aligned. The inner tube 512 and the outer tube 514, in the exemplary embodiment, are generally cylindrical in shape, having an approximately circular cross-section. One skilled in the art will appreciate, however, the inner tube 512 and the outer tube 514 may have other cross-sectional shapes, including, for example, elliptical or rectilinear. In the exemplary embodiment, the inner tube 512 and outer tube 514 have analogous cross-sections, however, one skilled in the art will appreciate that the inner tube 512 and the outer tube 514 can have different cross-sectional shapes. The axial length of the inner tube 512 and outer tube 512 may vary depending on, for example, the patient anatomy, the procedures employed, and/or, that area of the spine in which the device 510 is employed. The inner tube 512 and the outer tube 514 may be linear, as in the exemplary embodiment, or may curved or angled along one or more sections or the entire length thereof. The inner tube 512 and the outer tube 514 may be constructed from any suitable biocompatible material, including, for example, a metal, such as stainless steel, or a polymer, from any conventional method of manufacturing medical devices.

Although the illustrated exemplary embodiment includes an inner tube and an outer tube, one skilled in the art will appreciate that any number of tubes, e.g., one or more tubes, may be employed depending on, for example, the type of bone anchor employed and the manner by which the device is releasably engaged to the bone anchor.

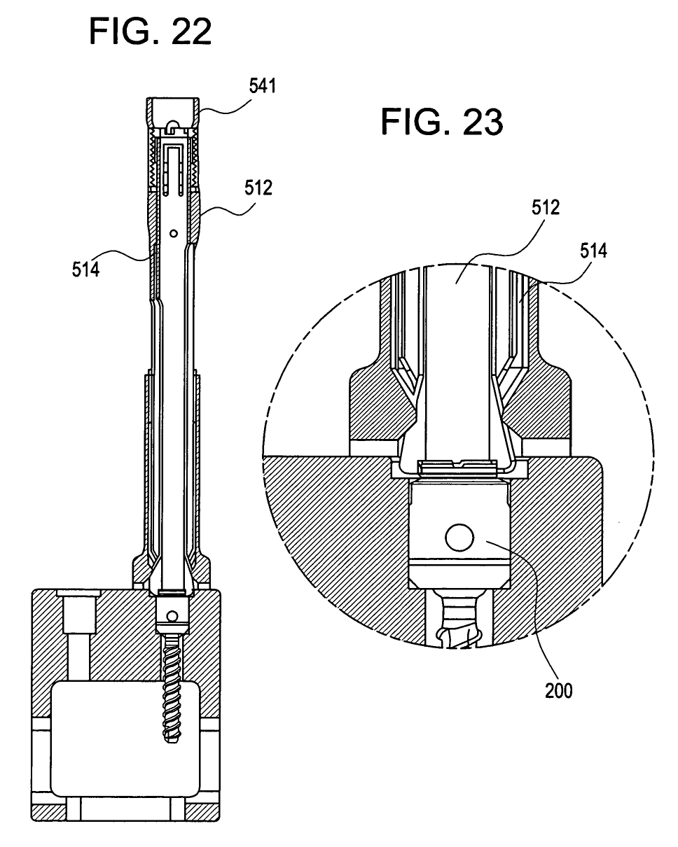

The inner tube or sleeve 512 includes a proximal end 520, a distal end 522, and a lumen 524 extending between the proximal end 520 and the distal end 522. The lumen 524 extends the length of the inner tube 512 and defines a longitudinal axis of the inner tube 512. The outer tube or sleeve 514 includes a proximal end 530, a distal end 532, and a lumen extending between the proximal end 530 and the distal end 532. The lumen of the outer tube 514 may extend the length of the outer tube 514 and defines a longitudinal axis of the outer tube 514. The inner tube 512 is positionable within the lumen of the outer tube 514. In the exemplary percutaneous access device 500, the inner tube 512 is longitudinally adjustable with respect to the outer tube 514. For example, the inner tube 512 may adjustable from a first, proximal position, in which the distal end 522 of the inner tube 512 is positioned proximal to the distal end 532 of the outer tube 514 as illustrated in FIG. 20, and a second, distal position, in which the distal end 522 of the inner tube 512 is positioned proximate to the distal end 532 of the outer tube 514. In the exemplary embodiment, the distal end 522 of the inner tube 512 preferably contacts at least a portion of the bone anchor assembly when the inner tube 512 is in the second position, as illustrated in FIGS. 22-24.

The exemplary percutaneous access device 500 may include an adjustment mechanism 540 that allows an operator to adjust the relative longitudinal position of the inner tube 512 and the outer tube 514. In the illustrated embodiment, for example, the adjustment mechanism 540 is a hollow, tubular shaped cap 541 having internal threads that engage external threads provided on the proximal end 530 of the outer tube 514. The threads allow the cap 541 to be longitudinal adjusted relative to the outer tube 514. In the exemplary embodiment, the inner tube 512 is connected to the cap 541 and, thus, can move longitudinally, but not rotationally, with the cap 541 as the cap 541 is advanced or withdrawn relative to the outer tube 514.

The inner tube 512 may have one or more sidewall openings or slots 560 formed therein. In the illustrated exemplary embodiment, the inner tube 512 includes two opposed slots 560 that extend longitudinally from the distal end 522 of the inner tube 512. Like the inner tube 512, the outer tube 14 may have one or more sidewall openings or slots 560 formed therein. In the illustrated exemplary embodiment, the outer tube 514 includes two opposed slots 560 that extend longitudinally from the distal end 532 of the outer tube 514. The slots 560 can be used to facilitate positioning of a spinal fixation device, such as a rod or a plate, relative to one or more bone anchors. In the illustrated exemplary embodiment, for example, the slots 560 can be aligned with the recess 214 in the receiving member 208 of the bone anchor assembly 200, when the percutaneous access device 500 is connected to and aligned with the bone anchor assembly 200, to facilitate the positioning of a spinal rod in the recess 214.

The percutaneous access device 500 is preferably releasably engageable to a bone anchor, such as bone anchor assembly 200. In the exemplary embodiment, the outer tube 514 may be releasably engaged to a bone anchor, such as bone anchor assembly 200. For example, the outer tube 514 may be engaged to a bone anchor in a manner that allows the percutaneous access device 500 to be connected to the bone anchor 200 during use, e.g., during implantation and/or delivery and/or fastening of a spinal fixation element to the bone anchor, and allows the percutaneous access device to be disconnected from the bone anchor 200 at the conclusion of the procedure. Preferably, the percutaneous access device 500 can be disconnected remotely. For example, the exemplary embodiment, the percutaneous access device 500 can be disconnected from the bone anchor by manipulation of the proximal end of the percutaneous access device 500.

The distal end 532 of the outer tube 514 includes a pair of opposed longitudinally extending tabs 570A and 570B that may releaseable engage a bone anchor. In the exemplary embodiment, the tabs 570A and 570B are defined by the sidewalls of the outer tube 514 and are separated by the slots 560. In certain exemplary embodiments, the tabs 570A and 570B may be flexible and resilient in the radial direction to facilitate connection to a bone anchor. For example, the tabs 570A and 570B may be flexed apart in the radial direction from a first, relaxed position to facilitate advancement of the tabs longitudinally over a portion of the bone anchor. Once positioned about a portion of the bone anchor, the tabs 570A and 570B may provide a radially compressive force on the bone anchor as the tabs 570A and 570B attempt to return to the first, relaxed position. In other exemplary embodiments, including the exemplary percutaneous access device 500, the tabs 570A and 570B need not be flexible and resilient.

In the illustrated exemplary embodiment, each tab 570A and 570B may include one or more radially inward facing projection 572 that is sized and shaped to seat within an opening provided in a portion of the bone anchor. The size, shape and number of projections can be varied depending on, for example, the opening(s) provided on the bone anchor and type of connection desired. In the illustrated exemplary embodiment, for example, each projection 572A, 572B is generally arcuate in shape and has a cross section that is complementary to an arcuate groove 230 provided in the spinal fixation element receiving member 208 of the exemplary bone anchor assembly 200 described above.

Continuing to refer to FIGS. 19-26, the distal end 522 of the inner tube 512 may include a contact surface 581 that contacts at least a portion of a bone anchor when the inner tube 512 is in the second position. In the illustrated exemplary embodiment, for example, the distal end 522 of the inner tube 512 may have two opposing generally arcuate contact surfaces 581. The contact surfaces 581, in the exemplary embodiment, are oriented approximately perpendicular to the longitudinal axis of the inner tube 512. In the illustrated exemplary embodiment, the contact surfaces 581 are configured to contact a generally arcuate contact surface provided on the proximal end of the receiving member of the exemplary bone anchor assembly 200. Preferably, the contact surface 81 is complementary in size, shape, and orientation to the contact surface on the bone anchor. One skilled in the art will appreciate that the configuration of the contact surface 581, e.g., number, size, shape, and orientation of the contact surface 581, may be varied to, for example, suit the bone anchor being employed.

The distal end 522 of the inner tube 512 and/or the distal end 532 of the outer tube 514 may be configured to inhibit rotation of the bone anchor assembly relative to the percutaneous access device 510. For example, the distal end 522 of the inner tube may include one or more finger-like extensions 582 that extend approximately axially from the distal end 522 of the inner tuber 12 and engage a bone anchor to inhibit rotation of the bone relative to the percutaneous access device. For example, one or more of the extensions 582 may seat within a groove, recess, slot, or similar structure provided in the bone anchor. Alternatively, one of more of the extensions 582 may include a contact surface 584 for contacting an axially extending surface of the bone anchor, as in the case of the exemplary embodiment.

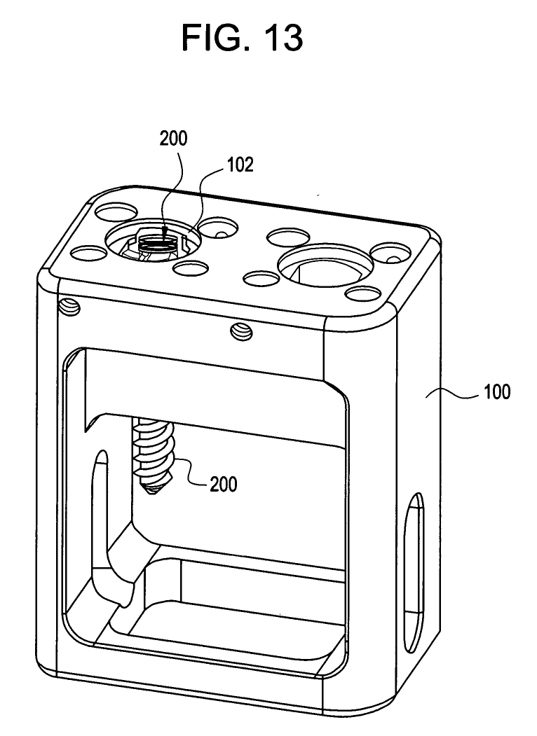

The alignment devices and guide tubes described herein may be employed to facilitate attachment of the exemplary percutaneous access device 500 to the exemplary bone anchor assembly 200. For example, the exemplary bone anchor assembly 200 may be positioned in the an anchor opening 102A of the alignment device 100, as illustrated in FIGS. 13-15. The proximal portion 130 of the anchor opening 102A receives the receiving member 208 of the bone anchor assembly 200 and the distal portion 132 of the anchor opening 102 receives the shank 206 of the bone anchor assembly 200. The grooves 230 provided on the receiving member 208 are positioned within the counter sink 140 of the anchor opening 102A. The size and shape of the anchor opening 102A positions the bone anchor assembly 200 such that the recess axis 232 defined by the recess 214 of the bone anchor assembly 200 is oriented generally parallel to the first axis 124 of the alignment device 100. In particular, the planar sides 230 of each leg 224A, 224B are aligned with the planar side walls 134 of the proximal portion 130 of the anchor opening 102. In the exemplary embodiment, the cross sectional size and shape of the proximal section 130 of the anchor opening 102 is analogous to the size and shape of the receiving member 208 of the bone anchor assembly 200 to inhibit rotation of the receiving member 208 within the anchor opening 102.

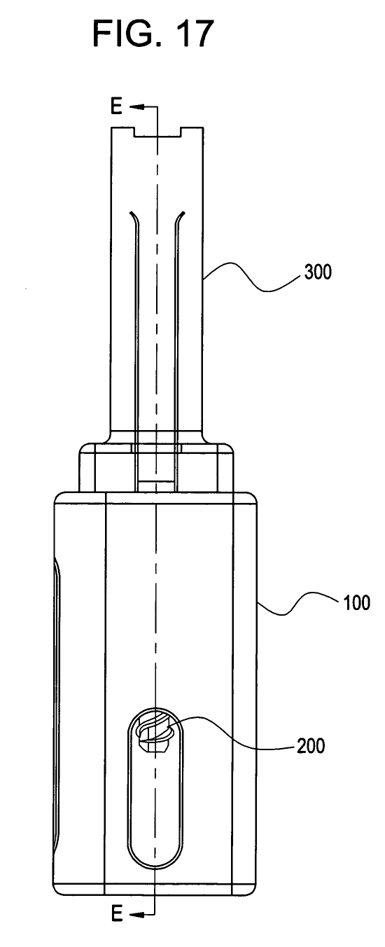

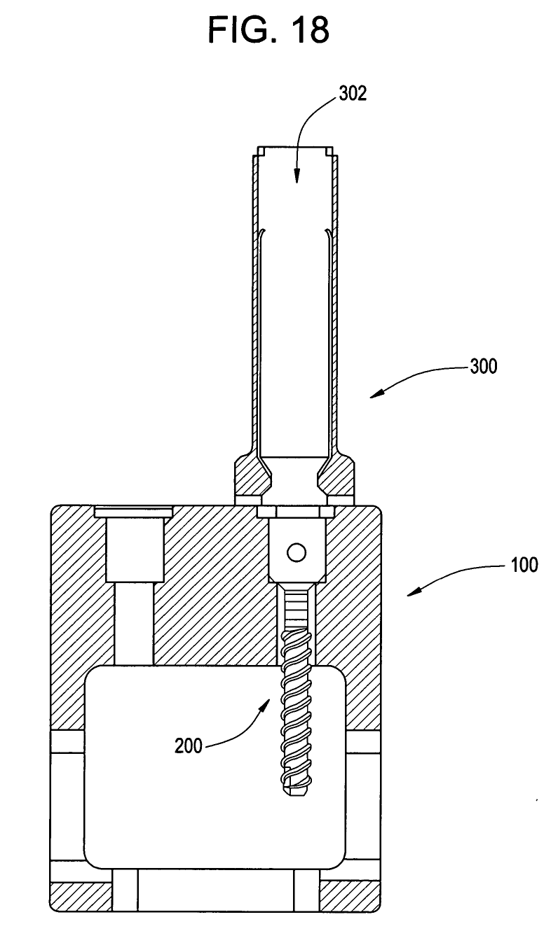

Referring to FIGS. 17 and 18, the guide tube 300 can be connected to the alignment device 100 and aligned such that the lumen 302 of the guide tube 300 is aligned with anchor opening 102A. To facilitate alignment of the guide tube 300 with the anchor opening 102, the alignment posts 350 are positioned within the respective connection openings 150 of the alignment device 100. As discussed above, the alignment posts 350 and the connection openings 150 cooperate to position the guide tube 300 relative to the alignment device 100 such that the tab axis 352 intersecting the tabs 320A, 320B is oriented parallel to the first axis 124 of the alignment device 100 and parallel to the recess axis 232 defined by the recess 214 of the bone anchor assembly 200.

Referring to FIGS. 19-21B, a delivery instrument, such as the exemplary percutaneous access device 500, may be positioned into the lumen 302 of the guide tube 300 in a first orientation. For example, the percutaneous access device 500, may be positioned into the lumen 302 of the guide tube 300 in the first, proximal position, described above, in which the distal end 522 of the inner tube 512 is positioned proximal to the distal end 532 of the outer tube 514 as illustrated in FIG. 20. As the distal end of the percutaneous access device 500 is advanced distally, the outer tube 514 abuts the engagement section 324 of each tab 320 and displaces the engagement sections 324 radially outward, as illustrated in FIG. 20. The percutaneous access device 500 can be advanced distally within the lumen 302 of the guide tube 302 until the distal end 532 of the outer tube 514 engages the counter sink 140 of the anchor opening 102. Once positioned, the lumen of the percutaneous access device 500 is aligned with the bone anchor assembly 200 positioned in the anchor opening 102. In this first orientation, a slot axis intersecting the diametrically opposed slots in the percutaneous access device 500 are oriented perpendicular to the first axis 124 of the alignment device 100 and perpendicular to the recess axis 232 defined by the recess 214 of the bone anchor assembly 200.

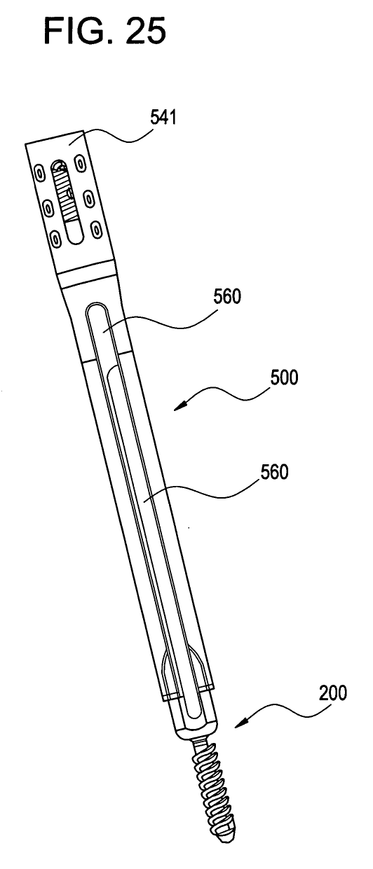

The percutaneous access device 500 can be connected to the bone anchor 200 by moving the percutaneous access device 500 to a second orientation. For example, the percutaneous access device 500 can be rotated to a second orientation in which the projections 572A, 572B of each tab 570A and 570B engage the grooves 230A,B of the receiving member 208 of the bone anchor assembly. As the percutaneous access device 500 is rotated, the engagement sections 324A,B each engage a slot 560 provided in the percutaneous access device 500 and, upon engagement of the slot 560, the engagement sections 324 move radially inward, returning the tabs 320 A,B to the first position. Once the engagement sections 324 are positioned in the slots, further rotation of the percutaneous access device 500 is inhibited. In the exemplary embodiment, the percutaneous access device 500 can be rotated from the first position, illustrated in FIG. 20, approximately 90° about the longitudinal axis of the percutaneous access device 500 to the second position, illustrated in FIGS. 22-24. In the second position, the slots 560 of the percutaneous access device 500 are aligned with the recess 214 of the receiving member 208 of the bone anchor assembly 200. The inner tube 512 may be advanced distally into contact with the bone anchor assembly 200 to complete attachment of the percutaneous access device 500 to the bone anchor assembly 200. The percutaneous access device 500 and bone anchor assembly 200 may be removed from the guide tube 300 and the alignment device 100, as illustrated in FIG. 25.

In certain exemplary embodiments, a bone anchor driver 600, such as a screw driver, may be positioned in the percutaneous access device 500 and engaged with the bone anchor assembly prior to removal of the percutaneous access device 500 and bone anchor assembly 200 from the guide tube 300 and the alignment device 100.

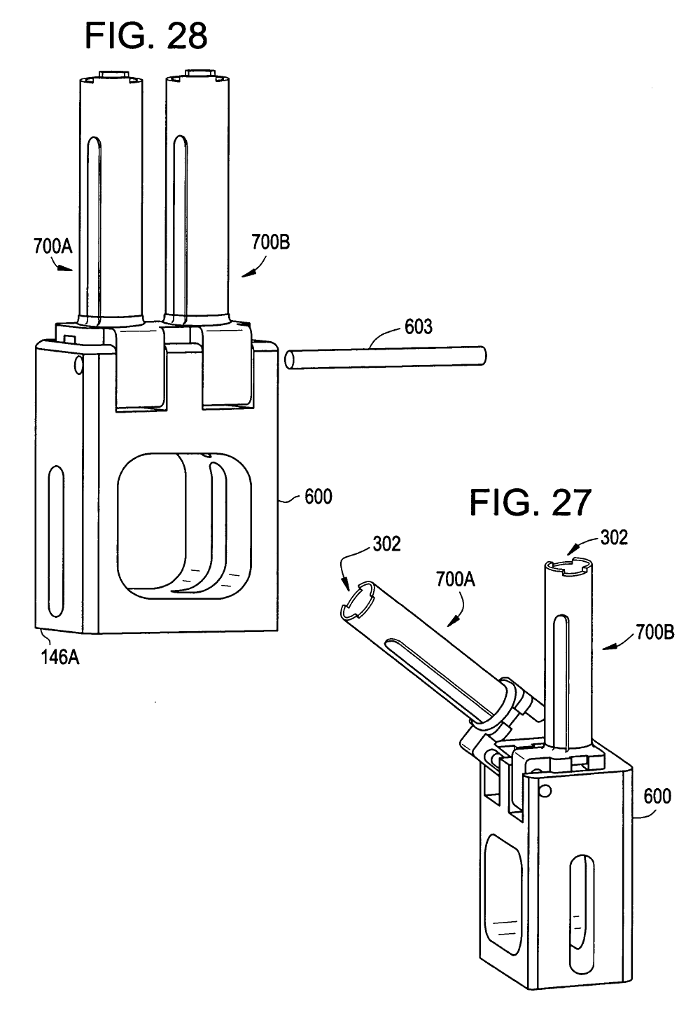

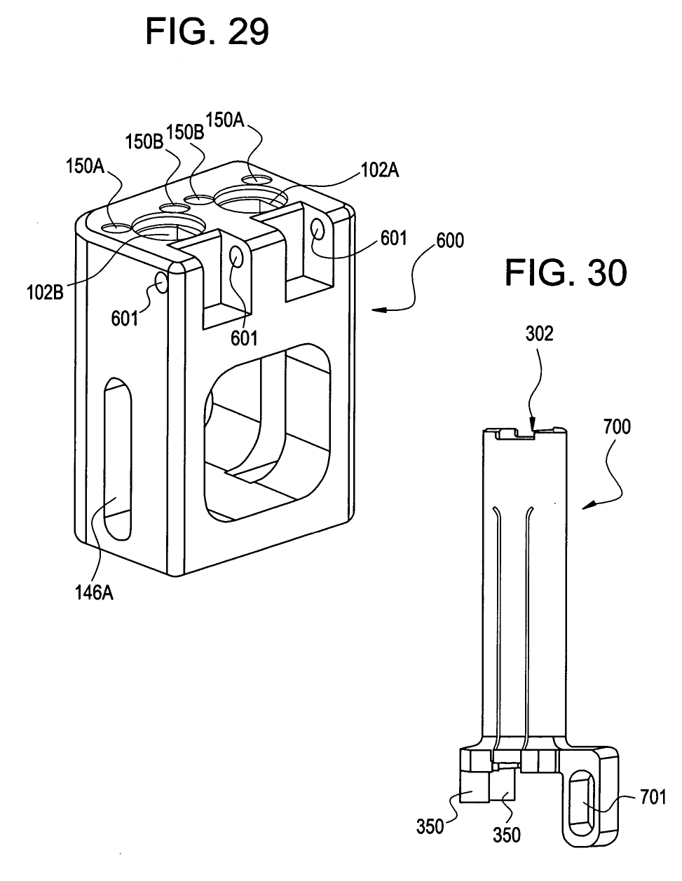

FIGS. 27-30 illustrate an alternative embodiment of an alignment device and one or more guide tubes that may be pivotally connected to the alignment device. In the illustrated exemplary embodiment, the one or more guide tubes 700 are pivotally connected to an alignment device 600. The guide tubes 700 and alignment device 600 may be constructed in a manner analogous to the guide tubes and devices described above. In the exemplary embodiment, the alignment device 600 includes one or more openings 601 and the guide tubes 700 include one or more openings 701 for receiving a shaft 603 about which the guide tube 700 may pivot or rotate. For example, a guide tube 700 may be pivoted between a first position in which the lumen 302 of the guide tube 700 is aligned with an anchor opening 102 in the alignment device 600 and a second position in which the lumen 302 of the guide tube 700 is not aligned with the anchor opening 102 in the alignment device 600. FIG. 27 illustrates a guide tube 700A in a second position and a guide tube 700B in a first position. In the exemplary embodiment, the distal end of the guide tube 700 includes an opening 701, in the form of a slot, for receiving shaft 603, and two spaced apart alignment post 350 for positioning in connection openings 150 positioned about an anchor opening 102.

In alternative embodiments, one or more of the guide tubes may be integrally connected to an alignment device.

While the alignment systems and methods of the present invention have been particularly shown and described with reference to the exemplary embodiments thereof, those of ordinary skill in the art will understand that various changes may be made in the form and details herein without departing from the spirit and scope of the present invention. Those of ordinary skill in the art will recognize or be able to ascertain many equivalents to the exemplary embodiments described specifically herein by using no more than routine experimentation. Such equivalents are intended to be encompassed by the scope of the present invention and the appended claims.

1. A system for facilitating attachment of a bone anchor with an instrument, the system comprising:
an alignment device including an anchor opening sized and shaped to receive a bone anchor in a predefined orientation, and
a guide tube connectable to the alignment device, the guide tube having a lumen for receiving a delivery instrument for the bone anchor, wherein the lumen of the guide tube is aligned with the anchor opening in the alignment device upon connection of the guide tube to the alignment device.

2. The system of claim 1, wherein guide tube includes a plurality of alignment posts at a distal end of the guide tube.

3. The system of claim 2, wherein alignment device includes a plurality of connection openings arranged about the anchor opening in the alignment device to facilitate connection of the guide tube to the alignment device.

4. The system of claim 3, wherein the plurality of connection openings include a first connection opening having a first extent and a second connection opening having a second extent distinct from the first extent.

5. The system of claim 3, wherein the plurality of connection openings include a first connection opening having a first shape and a second connection opening having a second shape distinct from the first shape.

6. The system of claim 1, wherein the plurality of alignment posts include a first alignment post having a first size and first shape and a second alignment post having a second size and a second shape, wherein at least one the second size and the second shape is distinct from the first size and the first shape, respectively.

7. The system of claim 1, wherein the guide tube includes a resilient tab for engaging a portion of the delivery instrument to facilitate rotation of the delivery instrument into engagement with the bone anchor.

8. A bone anchor delivery system comprising:
a bone anchor having a proximal receiving member for receiving a connection element and a distal shaft configured to engage bone;
a percutaneous access device connectable to the bone anchor;
an alignment device including an anchor opening sized and shaped to receive the bone anchor in a predefined orientation, and
a guide tube having a lumen for receiving the percutaneous access device, wherein the lumen of the guide tube is alignable with the anchor opening in the alignment device.

9. The system of claim 8, wherein the receiving member has a substantially U-shaped cross section defined by two legs separated by a recess for receiving the connection element, each leg being free at a proximal end of the receiving member.

10. The system of claim 9, wherein the anchor opening is sized and shaped to orient the recess of the receiving member of the anchor in a predefined orientation.

11. The system of claim 10, wherein the percutaneous access device comprises a tube having a slot formed therein, the slot opening at a distal end of the tube and extending toward the proximal end of the tube.

12. The system of claim 11, wherein the guide tube includes a resilient tab sized to engage the slot of the percutaneous access device to facilitate rotation of the percutaneous access device into a first orientation in which the slot of the percutaneous device is aligned with the recess of the receiving member.

13. The system of claim 12, wherein the guide tube guide tube includes a plurality of alignment posts at a distal end of the guide tube.

14. The system of claim 13, wherein alignment device includes a plurality of connection openings arranged about the anchor opening in the alignment device to facilitate alignment of the guide tube with the alignment device in a predefined orientation.

15. The system of claim 14, wherein the guide tube includes a pair of spaced apart resilient tabs, each tab being movable in a radially outward direction to an expanded configuration.

16. The system of claim 14, wherein the alignment posts and the connection openings are configured to align an axis intersecting the resilient tabs with an axis of the recess of the receiving member of the bone anchor when the bone anchor is positioned with the anchor opening.

17. The system of claim 8, wherein the guide tube is pivotally connected to the alignment device, the guide tube being pivotal between a first position in which the lumen of the guide tube is aligned with the anchor opening in the alignment device and a second position in which the lumen of the guide tube is not aligned with the anchor opening in the alignment device.

18. A method of connecting an instrument to a bone anchor, the method comprising
inserting a bone anchor into an anchor opening of an alignment device;
connecting a guide tube to the anchor device, a lumen of the guide tube being aligned with the anchor opening;
positioning a delivery instrument into the lumen of the guide tube in a first orientation; and
connecting the delivery instrument to the bone anchor by moving the delivery instrument to a second orientation.

19. The method of claim 18, further comprising rotating the delivery instrument relative to the bone anchor to connect the delivery instrument to the bone anchor.

20. The method of claim 19, wherein the anchor opening of the alignment device is sized to inhibit motion of the bone anchor relative to the alignment device.

21. The method of claim 19, further comprising rotating the delivery instrument approximately 90° about a longitudinal axis of the delivery instrument to connect the delivery instrument to the bone anchor.

22. A system for facilitating attachment of a bone anchor with an instrument, the system comprising:
an alignment device including an anchor opening sized and shaped to receive a bone anchor in a predefined orientation, and
a guide tube integrally connected to the alignment device, the guide tube having a lumen for receiving a delivery instrument for the bone anchor, wherein the lumen of the guide tube is aligned with the anchor opening in the alignment device.
